(19) United States
(12) Patent Application Publication
(10) Pub. No.: US 2004/0254594 A1
(43) Pub. Date: Dec. 16, 2004

(54) Cardiac defect occlusion device

(76) Inventor: Arthur Alfaro, Colts Neck, NJ (US)

Correspondence Address: DILWORTH & BARRESE, LLP, 333 EARLE OVINGTON BLVD., UNIONDALE, NY, 11553, US

(21) Appl. No.: 10/764,609

(22) Filed: Jan. 26, 2004

Related U.S. Application Data
(60) Provisional application No. 60/442,476, filed on Jan. 24, 2003.

(51) Int. Cl. A61B 17/08

(52) U.S. Cl. 606/151; 606/215

(57) ABSTRACT

An occlusion device for occluding an abnormal opening includes a body configured to receive expansion medium so as to expand to a deployed position, in which the periphery of the body sealingly presses against the wall of the abnormal opening.

### CROSS-REFERENCE TO RELATED APPLICATION

This application is based on claims priority to a U.S. Provisional Application Ser. No. 60/442,476 filed on Jan. 24, 2003 and fully incorporated herein by reference

### BACKGROUND OF THE INVENTION

1. Field of the Invention

The present invention relates to a device and method for the repair of intracardiac and vascular septal defects by percutaneous catheter placement of a corrective prosthetic device.

2. Description of the Related Prior Art

Either congenitally or by acquisition, abnormal openings or holes can occur between adjacent chambers of the heart or its associated major blood vessels. Such openings are referred to, respectively, as interatrial and interventricular septal defects or patent ductus arteriosus and aortico-pulmonary windows. Such openings cause blood to leak from one chamber or artery to another and result in decreased pumping efficiency of the heart. Similarly, if defects occur in the Foramen Ovale, such defects, referred to as Patent Foramen Ovale (PFO), may result in a cerebral embolism. These deformities usually are congenital, however, they can also occur following a heart attack, significantly complicating subsequent coronary treatment and recovery. Such defects typically impose added strain on the heart and ultimately may lead to heart failure if not corrected.

The foramen ovale is an opening that exists between the right and left atria, the two upper chambers of the heart. During the fetal period, this communication is necessary for blood to bypass the circulation of the lungs (since there is no air in the lungs at this time) and go directly to the rest of the body. Within the first few days of life, this opening seals, ending the link between these heart chambers. In approximately 25-30 percent of individuals, this communication persists as a small opening, called the PFO.

Traditionally, such defects have required extensive open chest surgical techniques for correction. Specifically, the repair of such defects required an open heart procedure in which the heart was exposed and then opened and the defect was sewn shut by direct suturing. In connection therewith, a patch of a synthetic prosthetic material such as Dacron, Teflon, silk, nylon or pericardium was used as a patch.

Although other methods of occluding defects, most notably the use of a plastic plug to occlude the defect, were suggested as early as the 1950s, such methods similarly require the use of open-heart surgery to access the defect and place the prosthetic implant.

Beginning in the early 1970s, a number of devices and methods were proposed for the percutaneous transluminal catheterization procedure for the repair of intracardiac defects. For example, U.S. Pat. No. 3,874,388 to King, et al., describes a device in which a pair of umbrella-like occluders are positioned on opposite sides of a defect and drawn and locked together at a central hub, which crosses the defect. The device is said to effectively occlude the defect. Although the King device and method proposed to eliminate the need to perform open-heart surgery, its use and structure were very complicated, in that generally they required the umbrella-like occluders to be opened manually once positioned at the defect.

Similarly, U.S. Pat. No. 4,007,743, to Blake, relates to an umbrella-like defect closure device having a plurality of elongated struts pivotally mounted to a central hub. Each pair of adjacent struts is interconnected by a strip formed of a foldable, resilient material which serves to automatically and resiliently open each umbrella-like element once such element is released from a protective sheath. As in the King patent, the device includes two separate occluders, which are locked together by a snap connection once each of the occluder segments has been individually positioned across the septal defect.

Among the problems encountered with occluder devices of the designs described by King and Blake, is that they tend to be relatively rigid. In other words, the designs allow very little relative motion between the individual occluder elements and thereby require that each of said elements be placed precisely prior to seating at the septal wall and interconnection. In addition, because the devices described in the King and Blake patents include such relatively rigid structures, the devices are not particularly well suited for applications in which there is a variation in wall thickness at the site of the defect or in which the defect does not run perpendicularly through the septal wall.

Still another defect closure device is described in U.S. Pat. No. 4,917,089 to Sideris. The Sideris patent relates to an apparatus and method for transvenous closure of a septal perforation in the heart. The closure apparatus comprises an occluder which is positioned on the distal side of the perforation and an occluder-holder which is positioned on the proximal side of the perforation and is connected to the occluder across the perforation by means of a so-called “button” closure. As in the earlier transluminally delivered occluders, the Sideris patent requires that device elements positioned on opposite sides of a septal defect are separately delivered to the site of the defect and connected to one another in situ.

Although the Sideris patent describes a device in which the occluder and occluder-holder are not rigidly interconnected, the Sideris device still requires that the occluder be placed precisely on the distal wall portion of the septum because defect occlusion is provided by a single occluder element. Thus, even though there is some amount of relative movement allowed between the occluder and the occluder-holder of the Sideris device, the arms of the occluder are rigid and the occluder section must be precisely positioned to cover the entire distal side of the defect in order to prevent blood from leaking through the defect and to prevent the device from becoming dislodged.

Furthermore, like the King and Blake devices, the Sideris device requires in situ assembly to create the interconnection between the occluder and the occluder-holder. Such an in situ assembly requirement complicates the occlusion procedure because it requires that the occluder be positioned precisely and then maintained in that position during the assembly step. Manufacturing of implants or occluders made from memory shape alloys, which are rather expensive materials, requires sophisticated, cost-prohibitive industrial equipment. In use, implantation of the majority of the known occluders is performed by sophisticated, and thus expensive, medical equipment. Finally, quite often, health and/or technical reasons dictate the necessity of removal of the implanted devices. This procedure, which can be both technically and medically challenging, also requires expensive retrieving equipment.

Another method of treating a cardiac defect is disclosed in U.S. Pat. No. 6,485,489 teaching an inflatable occlusion device provided with a plurality of abrasive members. Due to the inflation of the balloon in the foramen ovale, the abrasive members are forced into interior tissue of the foramen ovale. The operator of the device can then remove endothelial cells and create trauma to the area by rotating and moving the device. The balloon is then deflated and retracted from the surgical site. Once the area inside the patent foramen ovale has been traumatized, the body's healing mechanism begins and then replaces the traumatized tissue with scar tissue permanently sealing the foramen ovale. Over time, the foramen ovale becomes completely obliterated and turns into the normal fossa ovalis. The disclosed method has yet to produce sufficient clinical data.

Accordingly, there is a need for a defect occlusion device characterized by a simple structure and associated with a reliable, but simple implantation and removal surgical procedure.

### SUMMARY OF THE INVENTION

Effectively addressing this need, the invention discloses a method and occlusion device configured to be controllably expandable to close the undesired opening in the anatomical structure. Applicable to both human beings and animals and primarily directed to occlusion of an intracardiac defect, the inventive device can be successfully utilized in both an open-heart surgery and in a less invasive, percutaneous surgical procedure.

In accordance with one aspect of the invention, the inventive occlusion device is configured to expand in a deployed position so that it assumes the shape and dimension of the opening to be occluded and prevents blood passage between adjacent heart chambers, such as left and right atriums. Due to the inherent physical characteristics of fluid, the inventive device has a heightened resistance to mechanical fatigue resulting from stresses imposed on the occlusion device during its use. On the other hand, the use of fluid as an expanding medium enhances the ability of the inventive device to reliably compress opposite surfaces in vivo by having its sealing sides conform to the contours of the opposite surfaces of the anatomical structure.

Additionally, by selecting certain non-compliant or semi-compliant materials for use in the inventive device, the ability of the occlusion device to resist corrosion over an extended time period can be critically enhanced. The Anti-corrosion characteristics of the inventive device further enhance its ability to resist failure over prolonged use.

Advantageously, the inventive occlusion device is clothed in a bio-compatible material serving as a scaffold, for surrounding tissue tending to grow into the device to form a strong mechanical bond therewith. Epithelialization of the inventive device provides the device with improved mechanical stability and resistance to outer stresses.

Another aspect of the invention relates to a guide/delivery system configured to have a compact structure. The compactness of the inventive device gains a particular significance in heart surgery characterized by the limited space within which the surgeon can safely operate.

Still another aspect of the invention is concerned with a retrieval device configured to remove the implanted device from the site in a simple and efficient manner. Particularly associated with a percutaneous method, the implanted inventive device is configured with a magnetic means facilitating its detection by and attachment to the retrieval device in vivo. Accordingly, the inventive retrieval device simplifies the surgical procedure and minimizes the risk of undesirable effects typically associated with the known retrieving devices.

Yet a further aspect of the invention relates to a new method of operating the inventive occlusion device.

Thus, it is an object of the present invention to provide an occlusion device configured to treat a variety of intracardiac defects such as Patent Foreman Ovale (PFO), Ventricular and Atrial Septal Defects (VSD and ASD) and having a simple and cost-efficient structure.

It is another object of the present invention to provide an occlusion device operating on the principle of expansion produced by fluid, which is delivered into the occlusion device after the latter has been installed in vivo.

It is yet another object of the present invention to provide an occlusion device configured to develop a reliable mechanical bond with surrounding tissue.

It is a further object of the invention to provide a delivery/guide system associated with the inventive occlusion device and having a simple and compact structure.

It is another object of the invention to provide a retrieval device configured to easily detect and attach to the inventive occlusion device in vivo for the subsequent removal of the inventive occlusion device.

It is still another object of the invention to provide a method for operating the inventive occlusion device.

### BRIEF DESCRIPTION OF THE DRAWINGS

The above and other objects and features of the present invention will become apparent from the following detailed description when taken in connection with the accompanying drawings, wherein:

FIG. 1 is a schematic diagram of the fetal circulation of a mammal;

FIG. 2A is a view of an occlusion device configured in accordance with the invention and illustrated in a rest position;

FIG. 2B is a view of the inventive occlusion device shown in a deployed position;

FIG. 3 is a side, elevational view of a delivery assembly and a occlusion device illustrated in a deployed position.

FIG. 4 is an exploited side elevational view of a guide system associated with the delivery assembly and configured to guide the occlusion device towards the heart of the patient.

FIGS. 5A-5D preparatory steps of the inventive method;

FIG. 6 illustrates an insertion position of the guide system of FIG. 4 with the defect occlusion device shown in a partially deployed position

FIG. 7 is a view similar to FIG. 6 with the defect occlusion device shown in a fully deployed position.

FIG. 8 is a view of the defect occlusion device shown in its deployed position immediately after the guide system has been detached from the device.

FIG. 9 is a view similar to the one of FIG. 8 showing the final installment position of the inventive occlusion device;

FIG. 10 is a schematic view of a retrieval device configured in accordance with the invention.

FIG. 11 is a view of the retrieval device of FIG. 9 shown in an operative position.

FIGS. 12A-12B are schematic views illustrating consecutive stages of the removal of the inventive occlusion device.

FIG. 13 is a schematic view of another embodiment of the inventive occlusion device.

### SPECIFIC DESCRIPTION

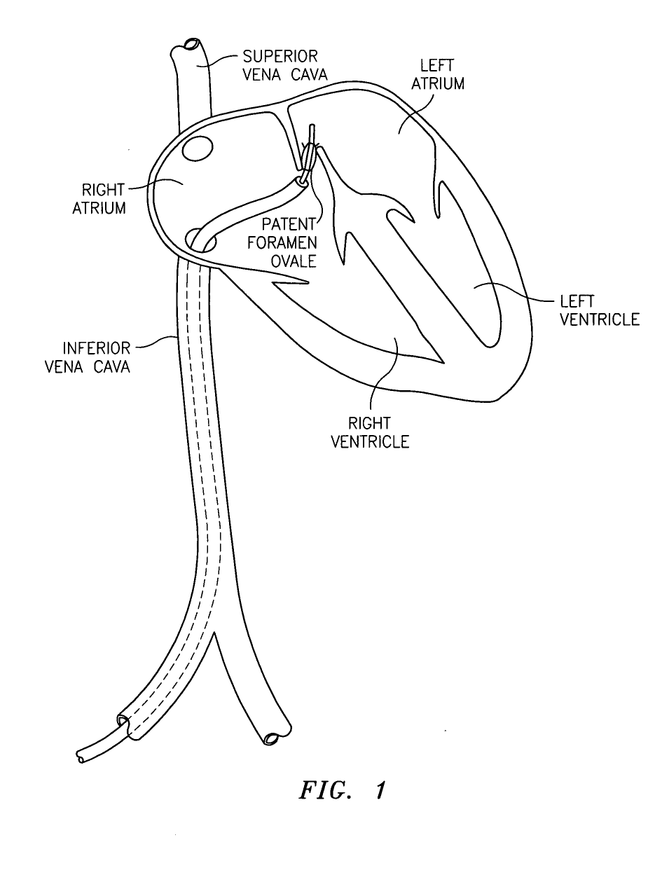

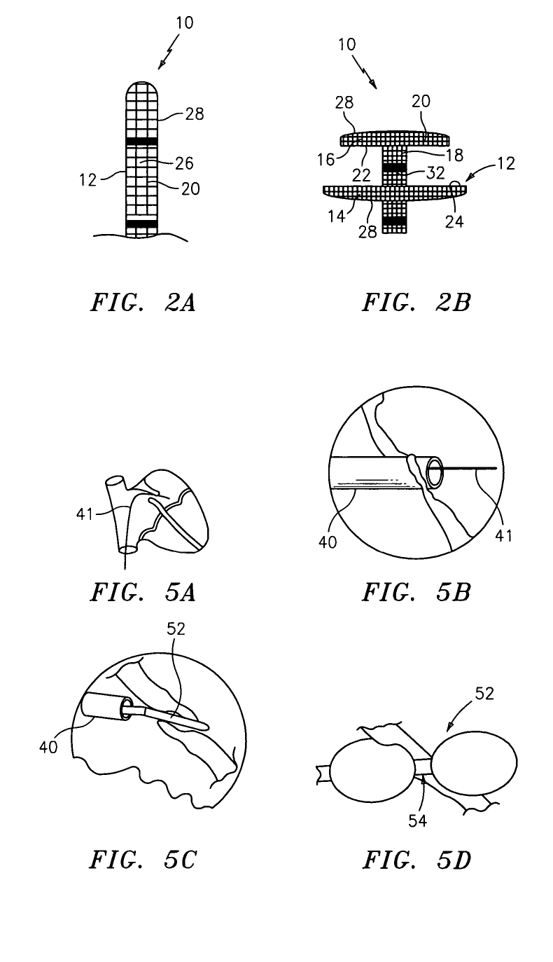

Referring to FIGS. 1 and 2A-2B, the present invention provides an occlusion device 10 configured to treat humans as well as animals suffering from various heart defects including, but not limited to, PFO, ASD, VSD, and PDA associated with undesirable openings in the heart. Purely for the illustrative purposes, the following description is mainly directed to the use of the inventive occlusion device 10 for closing a patent foramen ovale (PFO); however, the device 10 and the method of using this device can be invariably applied to treating other anatomical defects.

In particular, as illustrated in FIGS. 2A-2B, the occlusion device 10 is configured to receive fluid controllably expanding the device in the PFO to a degree sufficient to close the undesirable opening between the right and left atrium. The device 10 includes an expandable body 12, made from non-compliant, semi-compliant or compliant material and configured to receive fluid, and a bio-compatible material 20 providing for the ingrowth of the body 10 into surrounding tissue.

It is imperative that expansion fluid be biocompatible with blood. In other words, such a fluid should not pose any health-hazard if mixed with the blood of the patient. Water or saline mixed with radiopaque dye are well-tested liquids widely used in medical procedures. Alternatively, expansion gaseous medium used with a liquid.

The position of the inventive occlusion device 10 within the PFO in its deployed state and other necessary information can be obtained by using RF or ultrasound equipment. To effectively use the latter, an ultrasound agent is injected into the expansion medium to allow the effective use of ultra-sound equipment. Both blood-derived contrast agents and non-blood-derived agents are available for use with heart ultrasound technology. The latter may include ultrasound contrast agent Definity™ (DuPont Pharmaceutical Company). Still another contrast agent is air, known to reflect or scatter ultrasound much more strongly than body tissue. Using the known techniques, the air can be introduced into the expansion medium as microbubbles dissolving innocuously in time.

Expansion of the body 12 within the PFO blocks blood communication between adjacent atriums and brings a tissue flap, if one exists, into approximation of opposing tissue to allow healing and closure of the PFO. Generally, any compliant, semi-compliant and/or non-compliant bio-compatible materials 26 (FIG. 2A) can form the body 12. Advantageously, such materials are either non-compliant or semi-compliant and may include, among others, polyethylene terephthalate (PET), or a polyamide (non-compliant) material, or a radiation cross-linked polyethylene, polypropylene, polyethylene terephthalate (semi-compliant) material, and a combination of these materials. Other materials, such as latex, silicone, polyurethane and fluoro-elastomer can be used as well.

Non-compliant materials offer the advantages of a predictable size and pressure feedback when inflated. As is known, the size and shape of the PFO varies and, thus, requires differently shaped and sized implants or occlusion devices. In accordance with one aspect of the invention, a surgical kit, among other components, is configured to include numerous devices 10, each of which would have a predetermined shape and size in the deployed position.

Alternatively, the body 12 can be made from compliant materials, which inherently offer the advantages of variable size and shape conformance to adjacent tissue geometry. Consonant with the inventive aspect, in addition to the device made from non-compliant material or alternatively thereto, the surgical kit has at least one occlusion device 10 made of compliant material and capable of assuming different shapes and sizes depending upon the size and shape of the opening.

Compositionally, the body material 26 may have a pharmaceutically acceptable drug, such as an anti-inflammatory agent, anti-thrombotic, an anti-virus agent, an antibiotics, an anesthetic agent, and a combination thereof.

Depending on the thickness of the septum, the body 12 can be configured to have either a single neck portion extending within the opening to be occluded, as shown in FIG. 2A, or to assume an H-shape configuration in the deployed position, as illustrated in FIG. 2B. Other shapes could include, but not limited to, “C” or “U” shape, “T” Shape, “B” shape, “Z” or “N” shape, “P” shape, “Y” shape, or “X” shape. The body 12 of FIG. 2A may be more suitable for the ASD or VSD associated with a relatively thick portion of septum. The PFO is advantageously treated with the device 10 of FIG. 2B, which is configured with proximal and distal wide chambers 14, 16 and is shown in the deployed position of the device 10, wherein the chambers are expandable in the right and left atriums, respectively. Preferably, the proximal chamber 14, expandable in the right atrium, is larger than the distal chamber 16 because it is configured to cover a larger tissue surface in the right atrium and to block particularly dangerous blood flow from the right atrium to the left atrium. A narrow neck 18, extending through the PFO between the opposite ends/chambers 14, 16, is expandable in the deployed position of the device 10 so that it presses against the peripheral wall of the opening and, thus, blocks the blood passage between the atriums. Controllable expansion of the neck 18 as well as the chambers 14 and 16 can be obtained by installation of valves, as explained herein below.

One of the most challenging aspects of any implantation procedure is the ability of the implant to be incorporated in the surrounding tissue. Accordingly, in accordance with another aspect of the invention, epithalization of the occlusion device 10 is provided by bio-compatible material 20 configured to provide the ingrowth of tissue. Structurally, the material 20 can be formed as a separate outer element coupled to and covering the material 26 of the body 12 or as an integral part of this material. A variety of materials associated with the tissue ingrowth may include, but are not limited to, polyester, nylon, polypropylene, polyethylene, Dacron mesh (knitted or woven) polyurethane, and a combination of these.

In case of the structure provided with the separate layer of material 20, the latter is configured to expand simultaneously with the body 12 and either covers the entire body 12 (FIG. 2A), as a sock, or, preferably, is selectively attached to the selective portions thereof. In accordance with the inventive method, as will be explained herein below, the inner surfaces 22, 24 of the distal and proximal chambers 16, 14, respectively, are preferably free from the material 20 to minimize the risk of damaging the material 26 of the body 12. Another advantage of having the inner surfaces 22, 24 “cloth”-free includes the improved conformity of the inner surfaces 22, 24 to the contour of the septum in the vicinity of the PFO, which improves the immediate sealing ability of the device 10. Another advantage of having the inner surfaces 22, 24 “cloth”-free includes minimizing trauma or erosion of tissue. To accomplish it, these inner surfaces may have a smooth or lubricous surface to make it less traumatic. However, such a structure may cause slight relative displacement between the tissue and the device 10. Thus, based on the surgeon's decision, the device 10 may be selected with the inner surface 22, 24 and outer surfaces 28 of the chambers 14 and 16 having an improved frictional characteristics. This could be accomplished by utilizing cloth, coatings, or a textured surface. The textured surface may include formations in the shape of spheres, squares or diamonds. Outer surfaces 28 of the chambers 14, 16, fully covered by the material 20, allow the tissue to grow over and into the material 20 within several months. To provide selective attachment of the layer of material 20, a variety of pressure- or heat-sensitive adhering means can be strategically located either on the body 12 or on the material 20 and activated at an initial stage of surgery when the device 10 is preferably in its rest position.

In accordance with other configurations of the device 10, to integrate the material 20 into the body material 26, the former can be extruded together with the body material 26. Accordingly, since the material 20 is embedded into the body 12, it will not be accidentally detached therefrom. It is preferred that the commonly extruded materials 20 and 26 form the outer, exposed surfaces 28 of the device 10, whereas the inner surfaces 22, 24 are totally or partially free from the material 20.

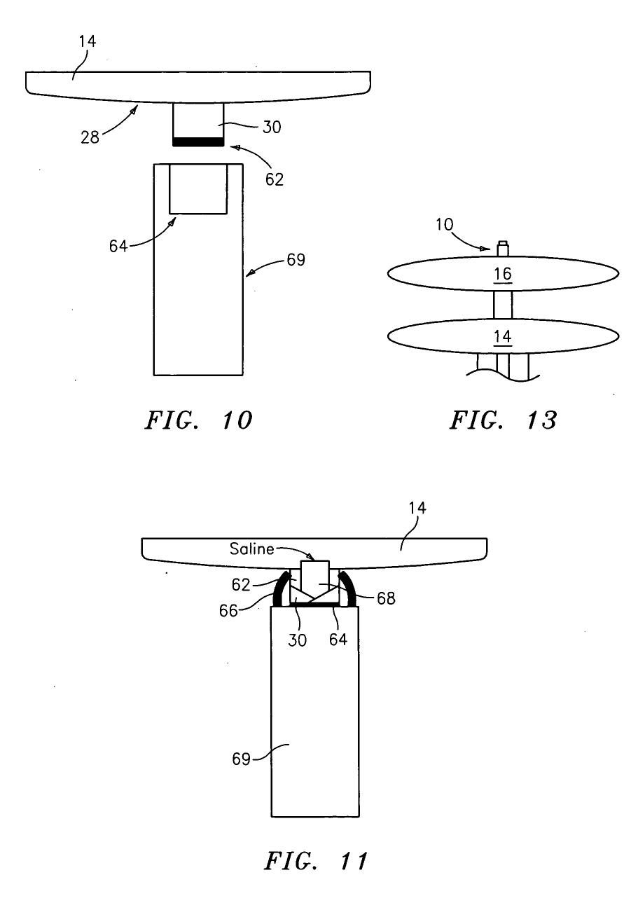

In light of diagnostic, surgical and safety reasons, it is imperative that an implant be traced during and after its installation. The use of fluoroscopic, ultra-sound and other techniques requires that the body material 26 and the cover material 20 include traceable elements. Desirably, such elements are characterized by radio-opaqueness allowing the operating surgeon to clearly see where the implant is located at any given stage of surgical or interventional procedure. As shown in FIG. 2B and further in FIG. 9, the occlusion device can be provided with at least one radiopaque band 32. In FIG. 2B, the device 10 has the band 32 located on the neck 18 substantially midway between its opposite proximal and distal ends. However, one or more radiopaque bands can be selectively positioned on the device 10 to provide the operating surgeon with clear view of any part of the inventive device. A variety of materials used for the identification purposes may include a group of metallic materials.

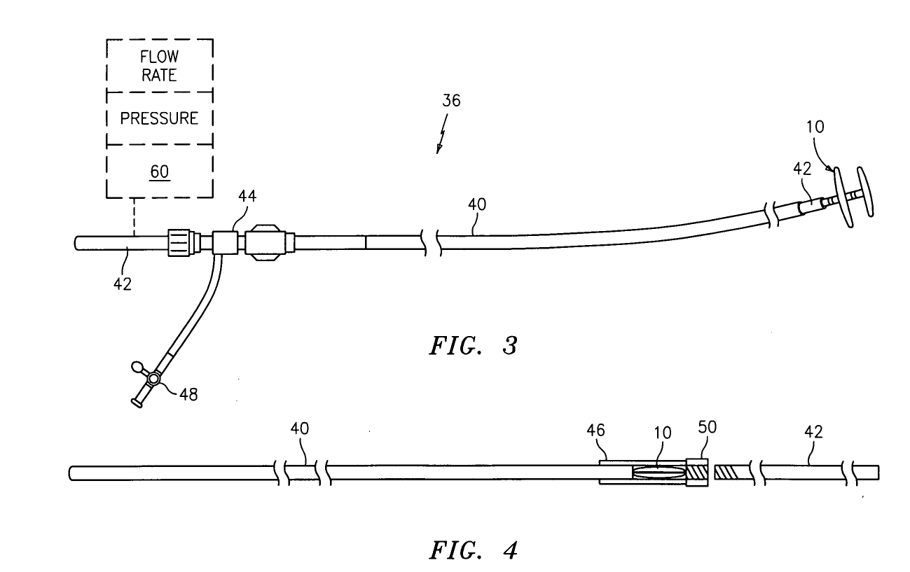

In accordance with a further aspect of the invention, the occlusion device 10 is provided with a means for its controllable expansion and deflation. Among others, such a means includes a one-way or two-way valve 30, as shown in FIG. 10. To provide proper functioning of the device 10, the valve 30 serves as inlet/outlet port into the chamber 14 expandable in the right atrium. However, to improve control over expansion of other portions of the device 10, such as the distal chamber 16, another two-way valve (not shown) can be installed between the latter and the neck 18. As will be shown, to facilitate the installation/removal of the device 10, the valve 30 may be provided with a variety of means facilitating detachable coupling between the valve and a guide/delivery assembly 36 for delivering the occlusion device 10 shown in FIGS. 3, 4.

In summary, the occlusion device 10 is associated with non-expensive materials and requires simple manufacturing equipment. Accordingly, the inventive device is cost efficient. During installation, the device 10 easily conforms to the variously dimensioned and shaped PFO and, upon expansion produced by expansion fluid, blocks the flow of blood between the atriums. Furthermore, provision of radiopaque bands and ultrasound scattering materials critically facilitates the implantation of the inventive device. As a result, the use of the inventive device simplifies the complicated surgical procedure. In addition, the device is readily fused with the surrounding tissue. Accordingly, the inventive device is characterized by its improved mechanical stability, although, if a need arises, it can be removed from the PFO.

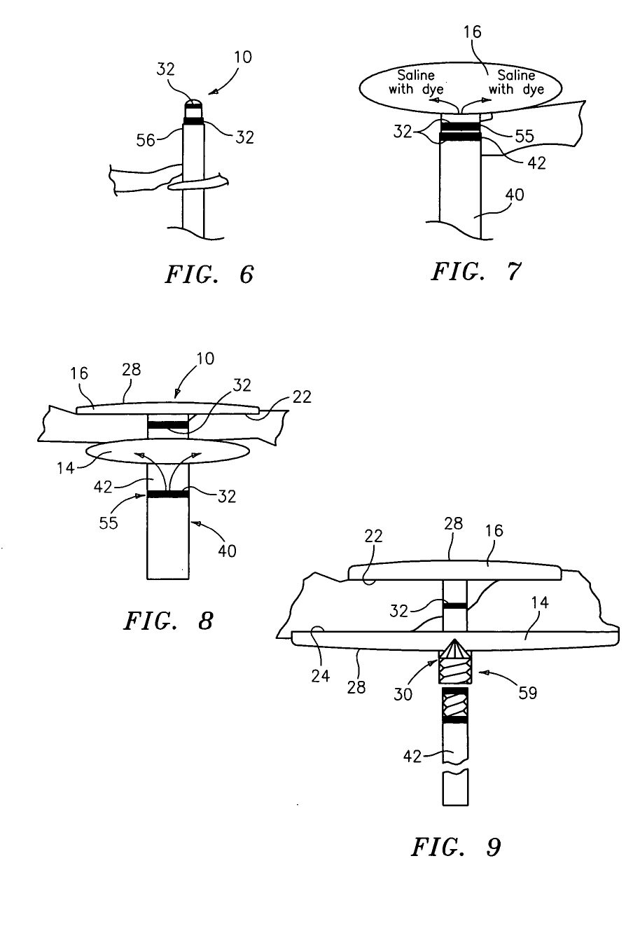

Turning to FIGS. 3-10, and particularly to FIGS. 3, 4, the inventive method of installing and operating the device 10 is associated with the delivery/guide assembly 36. Due to the limited operating space within the heart, the assembly 36 should be small and ergonomically configured. The assembly 36 includes a hollow guiding catheter 40 insertable across the PFO and dimensioned to be traversed by a cannula 42 having the device 10 preloaded therein. The proximal end of the assembly 36 is configured with a handle including a Borst/hemostatic valve 44, a controllably actuated flushing system 48 utilized to flush the assembly 36 during use to facilitate delivery of the implant and a transition delivery sheath 46. The cannula 42 is dimensioned to have a length sufficient to be greater than the distance between the insertion point of the device and the PFO and to extend beyond opposite proximal and distal ends of the assembly 36.

Advantageously, as shown in FIG. 4, the flexible guiding catheter 40 and the delivery sheath 46 are detachably coupled with one another by a threading arrangement 50 located just before the entry point into the body of the patient. The cannula 42 having its distal end preloaded with the occlusion device 10 is introduced through the delivery sheath and through the guiding catheter 40 to span the PFO. As will be explained below, after the device 10 is deployed across the PFO, the cannula 42 will convey the expansion fluid into the inventive device. The system allows the guiding catheter to be converted into a delivery catheter providing a larger working channel for the implant to be guided through.

Referring to FIGS. 5A-5D, the surgical procedure begins with scaling the PFO to be occluded. To complete this step, the guiding catheter 40 is introduced over a guide wire 41 through a small, about 1 inch, incision in the right leg into femoral vein and further advance to the right atrium across the PFO and is finally parked in the left atrium, as shown in FIG. 5B. Measurement of the PFO is accomplished by inserting a sizing balloon 52 (FIG. 5C) within the guiding catheter 40 over the wire 41 and subsequently inflating the sizing balloon 52. As shown in FIG. 5D, the diameter of the waist 54 of the sizing balloon 52 will determine the size of the PFO and the size of the device 10, which should be slightly larger than the size of the PFO to ensure the reliable lodging of the device 10 in the deployed position.

Referring to FIGS. 3, 4, 6, and 7 after the size of the device 10 has been determined, the sizing balloon 52 is withdrawn, and the cannula 42 (FIG. 3) is introduced through the handle of the assembly 36, the delivery sheath 46 (FIG. 4) into the guiding catheter 40. Advancement of the cannula 42 is stopped when its distal end, removably coupled to the preloaded device 10, extends into the left atrium. Distal ends 55 (FIG. 7) and 56 of the cannula 42 and guide catheter 40, respectively, each have radiopaque marker bands 32, such as metallic rings fluoroscopically detectable to facilitate navigation of these components and determination of their location.

After the guiding/delivery catheter 40 has been withdrawn into the right atrium, the position of the cannula 42 and the device 10 is determined based on the image of the radiopaque band 32 located on the device 10. Technically, the guiding catheter 40 is gradually retracted from the left atrium until the band 32 of the device 10 becomes visible under fluoroscopy. Assuming that the proper positioning has been established, and the band 32 is located within the PFO, expansion medium is delivered into the device 10 to expand only its distal chamber 16, since the proximal chamber 14 is still within the guiding catheter 40. Advantageously, the flow rate of the expansion fluid and the pressure inside the chambers are monitored by system 60 (FIG. 3), based on the previously determined size of the PFO and the material of the device 10. The flow rate and pressure are so selected and monitored that the chamber 16 assumes very low profile characterized by a slight curve on the outer surface 28.

Once the distal chamber 16 is expanded, the implant cannula 42 is gently retracted to assure maximal apposition, as shown in FIG. 8. In particular, in the desired position, the inner surface 22 of the distal chamber 16 is slightly impressed into the tissue to allow relatively easy growth of the tissue over the curved outer surface 28. Finally, the guiding catheter 40 is retracted further into the right atrium to allow the right or proximal chamber 14 of the device 10 to be controllably expanded, as shown in FIG. 9. Similarly to the distal chamber 14, the inner surface 24 of the proximal chamber 14 is impressed onto the tissue.

As the device 10 is positioned across the PFO and the opposite chambers are expanded to compress the tissue having the opening toward the main body of the organ, the cannula 42 is detached from the valve 30. While a variety of attaching arrangements can be implemented in the inventive structure, it is preferred that such an arrangement would have the valve 30 (FIG. 9) and a threaded safety attachment 59 formed on the distal end of the cannula 42. Finally, the catheter and the cannula are completely removed to leave the device 10, as illustrated in FIG. 9, fully implanted in the PFO.

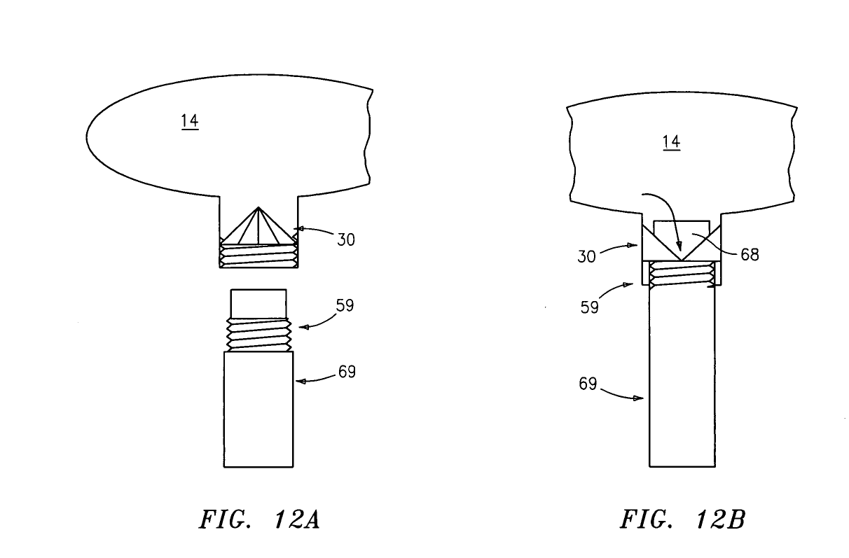

There may be an occasion when the physician would like to remove the occlusion device 10 after it has been deployed and the safety attachment 59 has already been disengaged. In this situation, a retrieval device 69 is deployed. In use, initially a retrieval device catheter (not shown) is snaked up to the right atrium. The catheter tip is docked over a valve nub 62, as illustrated in FIGS. 10-12. To facilitate this docking maneuver, the retrieval device 69 may contain a magnetically charged tip core 64 (FIG. 10), while the valve nub 62 is imbedded with ferrous metal. Conversely the valve nub 30 may contain a magnetically charged tip core while retrieval device 69 would contain imbedded ferrous metal or both the valve nub 62 and the retrieval device 69 could both contain magnetically charged material. After attachment has been completed, as shown in FIG. 11, grasping forceps 66 are displaced within the retrieval device 69 to grasp the implant valve nub 62. Finally, to evacuate expansion fluid from the device 10, a catheter 68 is displaced within the retrieval device 69 to enter the valve 30. As a result, once the occlusion device 10 has been deflated, the retrieval device 69 is removed from the site with the occlusion device 10 attached.

While the invention has been disclosed with respect to preferred embodiments, various changes can be made. For example, as shown in FIG. 13, the distal chamber 16 can be provided with a one-way valve formed on its outer surface 28. Such a valve can be accessed from outside to allow the entry into the left atrium. However all the changes can be made without departing from the scope of the invention as defined by the appending claims.

What is claimed is:

1. An occlusion assembly for occluding an abnormal opening in tissue comprising:
a collapsible body configured to receive an expansion medium to expand to a deployed position, in which the occluding device occludes the abnormal opening; and
at least one biomarker coupled to the collapsible body and operative to indicate a position of the collapsible body upon insertion thereof into the abnormal opening.

2. The occlusion assembly of claim 1, wherein the body has a neck and at least one at end portion provided with an inner surface which is juxtaposed with the tissue in the deployed position of the body.

3. The occlusion assembly of claim 2, wherein the body is made from material selected from the group consisting of compliant, non-compliant and semi-compliant material and a combination of these materials.

4. The occlusion assembly of claim 3, wherein the body material is selected from group consisting of terephthalate (PET), polyamide (non-compliant), radiation cross-linked polyethylene, polypropylene, polyethylene terephthalate, latex, silicone, polyurethane and fluoro-elastomer and a combination of these materials.

5. The occlusion assembly of claim 3, further comprising an ingrowth enhancing material selectively coupled to the body to enhance an ingrowth of the body into a surrounding tissue, the ingrowth enhancing material being selected from the group consisting of polyester, nylon, polypropylene, polyethylene, knitted, Dacron mesh, woven Dacron mesh, polyurethane, and a combination of these materials.

6. The occlusion assembly of claim 5, wherein the ingrowth enhancing material bio-compatible material covers an outer surface of the at least end portion spaced from the opening.

7. The occlusion assembly of claim 6, wherein the ingrowth enhancing material covers the outer surface of and an inner surface of the body, the inner surface of the body being adjacent to the opening in the deployed position of the body.

8. The occlusion assembly of claim 2, wherein the body further includes an additional end portion coupled to the neck and spaced from the at least one end portion so that the body has a dumbbell shape.

9. The occlusion assembly of claim 2, wherein the body further has a shape selected from the group consisting of an H cross-section, Z cross-section, N cross-section, C cross-section, U cross-section, T cross-section, B cross-section, Z cross-section, N cross-section, P cross-section, Y cross-section and an X cross-section.

10. The occlusion assembly of claim 8, wherein the body further comprising at least one valve located along a path of the expansion medium and configured to controllably provide ingress and egress of the expansion medium into and out of the body.

11. The occlusion assembly of claim 10, wherein the at least one valve is mounted to the additional end portion located upstream from the neck portion, the occlusion assembly further comprising an additional valve mounted on an outer side of the at least one end portion spaced downstream from the neck and the inner surface of the at least one end portion.

12. The occlusion assembly of claim 11, wherein the at least one and additional valves have a body provided with a threaded valve nab, which is operative to provide the egress of the working medium from the body when in the deployed position thereof.

13. The occlusion assembly of claim 12, further comprising a retrieval device configured to threadedly engage the valve nab of the at least one valve in the deployed position of the body, at least one of the valve nab and the retrieval device being imbedded with ferrous metal and the other one having a magnetically charged end portion to facilitate engagement between the valve nab and the retrieval device.

14. The occlusion assembly of claim 13, wherein the retrieval device has grasping forceps displaceable within the retrieval device to grasp the valve nub.

15. A method of occluding an abnormal opening formed in tissue, comprising the steps of:
(a) implanting an occlusion device in the opening; and
(b) delivering expansion fluid into the occlusion device so as to expand the occlusion device to a deployed position, wherein the occlusion device fills in the opening to prevent flow through the opening; and
(c) simultaneously with step (a), monitoring displacement of the expansion device.

16. The method of claim 15, wherein the occlusion device includes a neck portion bridging opposite end portion, the step (b) comprising sequentially expanding a remote end portion, the neck and a proximal end portion, thereby compressing the tissue to sealingly close the opening.

17. The method of claim 16, further comprising a step of providing the opposite end portions each with an ingrowth-enhancing material.

18. The method of claim 17, wherein the ingrowth-enhancing material selectively covers an outer periphery of the opposite end portions.

19. The method of claim 16, wherein the step monitoring includes tracing at least one fluoroscopic band provided on the body and controlling a rate of delivery of the working medium into the body and a pressure within the body to provide the body a low profile characterized by a slight curve on an outer surface of the body which is spaced fropom the opening.

20. A surgical kit comprising:
a delivery/guide assembly including an outer guiding catheter displaceable toward the opening and a cannula configured to be traversed by expansion medium and displaced through the guiding catheter to extend across the opening; and
a plurality of occlusion devices configured to have different shapes and dimensions and selectively and removably attachable to a distal end of the cannula, each of the occlusion devices being configured to receive the expansion medium and expand to a deployed position, wherein a periphery of the occlusion device sealingly presses against a wall of the opening.
